(19) United States
(12) Patent Application Publication
(10) Pub. No.: US 2021/0222123 A1
(43) Pub. Date: Jul. 22, 2021

(54) IN VITRO EXPANSION OF DOPAMINERGIC SUBTYPE NEURONAL PROGENITORS DERIVED FROM PLURIPOTENT STEM CELLS

(71) Applicant: Wisconsin Alumni Research Foundation, Madison, WI (US)

(72) Inventors: Su-Chun Zhang, Waunakee, WI (US); Xiang Li, Pacifica, CA (US)

(21) Appl. No.: 17/117,062

(22) Filed: Dec. 9, 2020

Related U.S. Application Data
(60) Provisional application No. 62/945,366, filed on Dec. 9, 2019.

(51) Int. Cl. C12N 5/0793; C12N 5/00

(52) U.S. Cl. CPC C12N 5/0619; C12N 5/0031; C12N 5/0056; C12N 2501/119; C12N 2500/90; C12N 2501/41; C12N 2501/727; C12N 2500/98; C12N 2501/415

(57) ABSTRACT

Methods and compositions for expanding dopaminergic neuron progenitor cells are described herein that include use of compositions and culture media that have at least the following components: an FGF, an agonist of SHH signaling, an agonist of canonical Wnt signaling, and Wnt-C59. The methods include contacting dopaminergic neuron progenitor cells with a culture medium comprising an FGF, an agonist of SHH signaling, an agonist of canonical Wnt signaling, and Wnt-C59, to generate an expanded dopaminergic neuron progenitor cell population.

### CROSS-REFERENCE TO RELATED APPLICATIONS

This patent application claims the benefit of priority of U.S. Provisional Patent Application No. 62/945,366, filed Dec. 9, 2019, which is incorporated herein by reference in its entirety.

### STATEMENT REGARDING FEDERALLY SPONSORED RESEARCH OR DEVELOPMENT

This invention was made with government support under NS076352 and NS096282 awarded by the National Institutes of Health. The government has certain rights in the invention.

### BACKGROUND

There are more than 1 million patients with Parkinson's disease (PD) in the US. PD results from loss of dopamine (DA) neurons in the midbrain. Current therapies, including the use of L-dopa and deep brain stimulation, treat symptoms but do not stop the disease progression. There is therefore a need to develop new therapies that stop/reverse the disease process or regenerate the lost neurons in PD. Human pluripotent stem cells (hPSCs) offer a promising source for generating authentic dopamine neurons for the development of disease-modifying therapeutics for PD. This may be achieved by building the human dopamine neuron-based drug discovery platforms for identifying medications that prevent or delay the DA neuron degeneration process or transplanting the DA neurons to replace the degenerated cells in PD patients.

Billions of DA neurons are needed for drug discovery through high throughput screening (HTS) or for cell transplantation therapy in hundreds or thousands of patients. One way to produce a large quantity of DA neurons is to start with large numbers of stem cells. Such a strategy would require DA neuron generation by many batches, which nevertheless creates variations among batches. An alternative is to expand the induced DA neuron progenitors by growth factors, but current methods for expanding fate-committed progenitors always result in loss of the original fate identity, i.e., loss of DA neuronal identity. In particular, the potential to produce large projection neurons such as midbrain dopamine neurons from induced DA neuron progenitors fades within two to four passages and is replaced by other neuronal populations.

Thus, there is an ongoing need for improved methods and compositions for producing large quantities of consistent DA neuronal progenitors suitable for differentiation into of dopamine neurons in volumes sufficient for high throughput screening and cell therapies.

### BRIEF SUMMARY

Described herein are methods, compositions, and kits that address the aforementioned drawbacks of conventional expansion protocols for dopaminergic neuron progenitors.

In a first aspect, provided herein is a method for expanding dopaminergic neuron progenitor cells. The method can comprise or consist essentially of contacting the dopaminergic neuron progenitor cells with a culture medium comprising fibroblast growth factor 8b (FGF8b), an agonist of Hedgehog (Hh) signaling, a small-molecule agonist of canonical Wnt signaling, and WNT-C59, to generate an expanded dopaminergic neuron progenitor cell population. The agonist of Hh signaling can be selected from the group consisting of Smoothened agonist (SAG), SAG analog, SHH, SHH C25II, C24-SHH, purmorphamine, Hg—Ag, and derivatives thereof. The small-molecule agonist of canonical Wnt signaling can be a glycogen synthase kinase 3 inhibitor. The glycogen synthase kinase 3 inhibitor can be CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof. The glycogen synthase kinase 3 inhibitor can be CHIR99021 and can be present in the culture medium at a concentration of about 0.01 micromolar (μM) to about 1 millimolar (mM). CHIR99021 can be present in the culture medium at a concentration of about 0.6 μM. WNT-C59 can be present in the culture medium at a concentration of about 0.2 micromolar (μM) to about 2 μM. WNT-C59 can be present in the culture medium at a concentration of about 0.5 μM. The dopaminergic neuron progenitor cells can expand in vitro at least 300-fold. The culture medium can further comprise neural supplement B27. The dopaminergic neuron progenitor cells can expand in vitro at least 1000-fold. In some embodiments, the culture medium comprises about 50 ng/ml FGF8b, about 25 ng/ml SHH, about 0.6 μM CHIR99021, and about 0.5 μM WNT-C59. The dopaminergic neuron progenitor cells can be sub-cultured at least 6 times without loss of phenotype or genotype. The culture medium can be chemically defined, serum-free, and xenogeneic material-free.

In another aspect, provided herein is a substantially pure population of human dopaminergic neuron progenitor cells obtained according to a method of this disclosure.

In a further aspect, provided herein is a composition comprising FGF8b, an agonist of Hh signaling, a small-molecule agonist of canonical Wnt signaling, and Wnt-C59. The composition can further comprise B27. In some embodiments, the composition consists essentially of FGF8b, an agonist of Hh signaling, a small-molecule agonist of canonical Wnt signaling, and Wnt-C59. In some embodiments, the composition consists essentially of FGF8b, an agonist of Hh signaling, a small-molecule agonist of canonical Wnt signaling, Wnt-C59, and B27. The small-molecule agonist of canonical Wnt signaling can be a glycogen synthase kinase 3 inhibitor selected from CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, and a lithium salt, or a combination thereof. The agonist of Hedgehog (Hh) signaling can be selected from the group consisting of Smoothened agonist (SAG), SAG analog, SHH, SHH C25II, C24-SHH, purmorphamine, Hg—Ag, and derivatives thereof. The composition can be formulated as a cell culture medium.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention and to cover all modifications, equivalents and alternatives. Reference should therefore be made to the claims recited herein for interpreting the scope of the invention.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

This invention will be better understood and features, aspects and advantages other than those set forth above will become apparent when consideration is given to the following detailed description thereof. Such detailed description makes reference to the following drawings, wherein:

FIG. 1A-1E demonstrate efficient generation of dopaminergic neuronal progenitors and neurons. (FIG. 1A) Schematic showing the process of deriving DA NPCs from hPSCs. (FIG. 1B) DA NPCs co-express signature DA transcription factors. (FIG. 1C) DA NPCs further mature into DA neurons. (FIG. 1D) Inducing efficiency of DA neurons from hPSCs. (FIG. 1E) Functional properties of DA NPCs-derived DA neurons.

FIG. 2A-2G demonstrate identifying FGF8 and SHH to expand DA NPCs. (FIG. 2A) Schematic outlining the process for testing compounds that promote DA expansion. (FIG. 2B) Quantification of absorbance with increased cell number ((Readout-Baseline)*10). (FIG. 2C) Evaluation of well-to-well and plate-to-plate variation. (FIG. 2D) Quantification of cell proliferation at different combinations of concentrations of SHH and FGF8 (Absorbance: (Readout-Baseline)*10). (FIG. 2E) Cell staining showing maintenance of DA signature markers after treatment at different combinations of concentrations of SHH and FGF8. (FIG. 2F) Maintaining DA identity and developmental potential after expansion. (FIG. 2G) Maintaining DA identity and developmental potential in a limited passage number.

FIG. 3A-3D demonstrate the identification of additional candidate by small molecule-based screening. (FIG. 3A) Schematic outlining the flowchart of chemical screening and validation of candidate compounds. (FIG. 3B) Plots displaying small molecule library top hits for inducing DA cell proliferation. (FIG. 3C) Cell staining for DA markers reveals WNT-C59 as the validated candidate from the small molecule library screen capable of expanding the cell population while maintaining DA identity. (FIG. 3D) Quantification of DA population expansion when treated with different combinations of small molecule cocktails and increasing concentrations of WNT-C59.

FIG. 4A-4E demonstrate that expanded DA NPCs retain DA identity and further mature into DA neurons. (FIG. 4A) Schematic showing the efficient expansion of DA NPCs using the FSCWB cocktail. (FIG. 4B) Generating large quantity of DA NPCs by using the method. (FIG. 4C) Cell staining showing DA identity was maintained during the expansion. (FIG. 4D) Further characterizing P6 DA NPC. (FIG. 4E) Quantification of expanded DA NPCs and their developmental potential to be matured into DA neurons at different passages.

FIG. 5A-5H present electrophysiological analysis for DA NPCs-derived neurons. (FIG. 5A) Voltage gated inward and outward currents with enlarged view of inward sodium currents in both P1 and P6 dopaminergic neurons. (FIG. 5B) I-V curve for P1 and P6 neurons. (FIG. 5C) Spontaneous firing of Action potentials in P1 and P6 neurons. Neurons were held at 0 pA and recorded continuously for 30 minutes to monitor sAP firing. (FIG. 5D) Evoked action potential: neurons were injected with 30 pA of current for 1 second resulted in evoked action potentials in both P1 and P6. (FIG. 5E) Evoked action potentials were observed in both P1 and P6 derived neurons by injecting with a series of 1 sec current steps ranging from −5 pA to 65 pA. (FIG. 5F-5H) membrane capacitance (Cm), membrane resistance (Rm) and resting membrane potential are similar for both P1 and P6. No significant changes were observed (unpaired t-test, P>0.05).

FIG. 6A-6D present RNA-seq analysis for expanded DA NPCs. (FIG. 6A) Principal component analysis of expanded DA NPCs. (FIG. 6B) Analysis of signature markers of subtype neuronal progenitors. (FIG. 6C) GO analysis of most changed gene expression. (FIG. 6D) Hierarchal clustering results of expanded DA NPCs. PSCs: pluripotent stem cells; FNPCs: forebrain neuronal progenitor cells and scMNPCs: spinal cord motor neuronal progenitors.

FIG. 7A-7C demonstrate transplantation of expanded DA NPCs and behavioral recovery in PD mice. (FIG. 7A) Scheme of transplantation and behavior test of PD mice model. (FIG. 7B) Histology analysis shows the majority of human cytoplasm expressing fibers co-expressing TH in the brain of PD model mice. (FIG. 7C) Quantification of cylinder and amphetamine induced rotation behavior tests at different time points post transplantation. In both embodiments a significant improvement was observed 5 months post transplantation.

FIG. 8 is a schematic illustrating the process of deriving DA neurons from hPSCs.

FIG. 9A-9D present characterization of DA identity maintenance during FGF8 and SHH optimization. (FIG. 9A) Testing different dose of FGF8 and SHH for DA expansion. (FIG. 9B) Cell staining showing that DA identity is maintained while using optimized concentrations of expansion compounds FGF8 and SHH. (FIG. 9C-9D) Cell staining showing that expanded DA NPC maintain their developmental ability to mature into DA neurons.

FIG. 10A-10B demonstrate testing FGF2 and CHIR99021 for DA expansion potential. (FIG. 10A) Testing different dose of FGF2 for DA expansion. (FIG. 10B) Testing different dose of CHIR for DA expansion.

FIG. 11A-11B demonstrate expansion of DA NPCs is enhanced when using chemical cocktail containing B27. (FIG. 11A) The addition of B27 to the FSCW cocktail further enhances expansion potential 3-fold. (FIG. 11B) The use of the FSCWB cocktail during passage yields increased cell number.

FIG. 12 presents quantification of amphetamine induced Rotation Test.

While this invention is susceptible to various modifications and alternative forms, exemplary embodiments thereof are shown by way of example in the drawings and are herein described in detail. It should be understood, however, that the description of exemplary embodiments is not intended to limit the invention to the particular forms disclosed, but on the contrary, the intention is to cover all modifications, equivalents and alternatives falling within the spirit and scope of the invention as defined by the appended claims.

### DETAILED DESCRIPTION

This invention provides improved methods for expanding human stem cell-derived DA neuronal progenitors, in certain embodiments, by 1000-fold within 30 days. As demonstrated in this disclosure, chemical screens and attribute testing were used to identify small molecules that provided a small benefit for expanding DA neuronal progenitors, and then those identified small molecules were tested in combinations to identify those that provided much greater effects in combination. The DA neuron progenitors expanded in the presence of the chemical cocktail described in this application retain the same DA neuron identity and possess the same therapeutic potency as the starting material when tested in the best-available mouse model of Parkinson's disease. By using this cocktail, a batch of 1-10 million DA neuron progenitors will produce 1-10 billion cells, sufficient for high throughput screening or cell therapy. These methods and compositions allow for unprecedented in vitro expansion of midbrain dopamine neural progenitors by 1000-fold in a few weeks.

These methods are a significant advancement over current state-of-art methods. In particular, these methods can generate consistent, sufficiently large populations of DA neurons for cell replacement therapy, particularly when a small number of DA neuron progenitors are purified by FACS. The expansion methods also make it possible to generate consistent, sufficiently large populations of DA neurons for modeling DA degenerative diseases and for high-throughput screening of DA neurons for drug discovery and validation.

Accordingly, in a first aspect, this disclosure provides in vitro methods for expanding dopaminergic neuron progenitors (DA NPCs), advantageously DA NPCs suitable for use in drug screening applications and for regenerative cell therapies. The methods enable scalable, industrial production of enriched or purified human DA NPCs. In exemplary embodiments, these methods comprise contacting human dopaminergic neuron progenitors in vitro culture with a chemical cocktail comprising a plurality of small molecules or other chemical compounds that promote proliferation of DA NPCs while maintaining dopaminergic identity and the capacity for differentiation into functional dopaminergic neurons. In some embodiments, the plurality of small molecules or chemical compounds includes an fibroblast growth factor (FGF), an agonist of the Hedgehog signaling pathway (also referred to as the Sonic Hedgehog “SHH” signaling pathway), an agonist of the canonical Wnt/β-catenin signaling pathway, and WNT-C59 (a potent inhibitor of Porcupine (PORCN), which is a key modulator of Wnt signaling). In some embodiments, the plurality further comprises B27, which is a serum-free nutritional supplement that promotes long term survival of in vitro cultured neurons.

In some embodiments, the plurality of small molecules or chemical compounds is a cocktail comprising the following agents: FGF8, SHH, CHIR99021, and WNT-C59. This combination of small molecules or chemical compounds is referred to herein as “FSCW cocktail” or “FSCW.”

In some embodiments, the FSCW cocktail further comprises B27 to form “FSCWB cocktail.” B27 supplement is available from various commercial vendors such as ThermoFisher. In some embodiments, FSCWB cocktail refers to a chemical cocktail comprising the following small molecules or chemical compounds: FGF8b, SHH, CHIR99021, WNT-C59, and B27.

As used herein, the term “dopaminergic neuron progenitors” or “dopaminergic neuron progenitor cells (DA NPCs)” refers to a progenitor or precursor cell which will mature, or is capable of maturing, into a dopaminergic neuron. Advantageously, the terms refer to a subpopulation of neural progenitor cells that can form a substantially homogenous cell population of midbrain dopaminergic neurons. Dopaminergic neuron progenitors are characterized by high levels of expression of OTX2, FOXA2, and SOX6 (a critical determinant for the development of midbrain DA neurons) as well as expression of other hallmark dopaminergic neuron genes including LMX1A, EN1, and CORIN, but substantially no expression of forebrain, spinal cord, or hindbrain markers.

As used herein, the term “expand” and grammatical variations thereof refer to inducing proliferation of a population of cultured dopaminergic neuron progenitors for at least 2, 3, 4, 5, 6, or more passages, or at least 2, 3, 4, 5, 6, or more weeks, without a change in cell identity and without loss of either DA neuron progenitor identity or capacity for differentiation into functional DA neurons. Expansion encompasses cell proliferation without differentiation or loss of cell identity or differentiation potential. As described in the Examples, DA NPCs cultured in the presence of FSCW and FSCWB cocktails can be expanded by 1000 fold over 6 passages without differentiation and without significant loss of DA identity while retaining the potential to generate functional DA neurons. Retention of DA identity can be assessed by any appropriate method including, without limitation, detecting expression of biomarkers of dopaminergic neuron progenitors such as OTX2, FOXA2, SOX6, LMX1A, EN1, and CORIN, and confirming the absence of expression of forebrain, spinal cord, or hindbrain markers. Neuronal function can be assessed using any appropriate method such as whole-cell patch-clamp recordings. DA function can also be assessed by determining the ability of transplanted expanded DA NPCs to rescue motor deficits in a mouse model of Parkinson's Disease.

Advantageously, the FGF is FGF8b or a derivative and/or variant thereof, wherein each derivative and/or variant thereof possesses one or more SHH signaling activator activities. In some embodiments, FGF8b is present in the culture medium at a concentration of about 25 ng/ml to about 200 ng/ml (e.g., about 25, 50, 75, 100, 125, 150, 175, 200 ng/ml).

Advantageously, WNT-C59 present in the culture medium at a concentration of about 0.2 micromolar (μM) to about 10 μM (e.g., about 0.2 μM, 0.3 μM, 0.4 μM, 0.5 μM, 0.6 μM, 0.7 μM, 0.8 μM, 0.9 μM, 1 μM, 2 μM, 3 μM, 4 μM, 5 μM, 6 μM, 7 μM, 8 μM, 9 μM, 10 μM). In some embodiments, WNT-C59 is present in the culture medium at a concentration of about 0.5 μM.

Any agonist of the canonical the Hedgehog (Hh) signaling pathway can be used. Exemplary Hg signaling agonists include, without limitation, Smoothened agonist (SAG), SAG analog, SHH, C25-SHH, C24-SHH, purmorphamine, Hg—Ag, and a derivative and/or variant thereof, wherein each derivative and/or variant thereof possesses one or more SHH signaling activator activities. In some embodiments, the agonist of Hh signaling is recombinant Sonic Hedgehog (SHH) or variant thereof. In some embodiments, the agonist of Hh signaling is a small-molecule such as purmorphamine, SAG, GSA-10,20(S)-hydroxycholesterol [20(S)—OHC], or a derivative or variant thereof. Purmorphamine is available from several commercial chemical compound vendors (e.g., Tocris Bioscience, Stemgent). Purmorphamine activates the Hedgehog (Hh) signaling pathways by directly targeting Smoothened (“Smo”), a critical component of the Hh signaling pathway. Sinha et al., Nature Chem. Biol. 2:29-30 (2006). Due to its toxicity, however, purmorphamine is less preferred to other agonists of Hh signaling. Other small molecule agonists of Smo which can be used to activate Hh signaling include, for example, SAG (“Smoothened Agonist”). The Hh pathway agonist SAG is a cell-permeable chlorobenzothiophene compound that modulates the coupling of Smo with its downstream effector. In some embodiments, the agonist of Hedgehog (Hh) signaling is the quinolinone GSA-10 (Hadden et al. (2014) ChemMedChem 9:27-37) or a synthetic oxysterol (OHC) such as 20(S)-hydroxycholesterol [20(S)-OHC]. OHCs can act on the cysteine-rich domain located in the Smo extracellular domain (ECD) to positively modulate Hh signaling.

Where the Hh pathway agonist is recombinant SHH or a variant thereof, the culture medium advantageously comprises an amount between about 10 ng/ml and about 100 ng/ml SHH, and more advantageously comprises about 25 ng/ml to about 50 ng/ml SHH. Accordingly, recombinant SHH can be contacted with DA NPCs at a final concentration in an in vitro culture of from about 10 ng/ml and about 100 ng/ml SHH, and more advantageously at a final concentration from about 25 ng/ml to about 50 ng/ml SHH.

Any small-molecule agonist of the canonical Wnt/β-catenin signaling pathway can be used. In some embodiments, the Wnt/β-catenin signaling pathway agonist is a GSK3 inhibitor. By inhibiting GSK3, CHIR99021 activates the canonical Wnt signaling pathway. CHIR99021 has been reported to inhibit the differentiation of mouse and human embryonic stem cells (ESCs) through Wnt signaling. See Wray and Hartmann, Trends in Cell Biology 22:159-168 (2012). Another GSK3 inhibitor that can be used is, for example, the Wnt/β-catenin signaling agonist 6-bromo-iridium-3′-oxime (“BIO”). See Meijer et al., Chem. Biol. 10(12):1255-66 (2003). GSK3 inhibitors such as those described herein are available from commercial vendors of chemical compounds (e.g., Selleckchem, Tocris Bioscience). In some embodiments, the GSK3 inhibitor is CHIR99021 or 6-bromo-iridium-3′-oxime. In some embodiments, the agonist is CHIR99021 (CHIR). Where the agonist is CHIR99021, the culture medium advantageously comprises about 0.01 micromolar (μM) to about 1 millimolar (mM) CHIR99021, and more advantageously comprises about 0.6 μM CHIR99021.

In some embodiments, the plurality of small molecules or chemical compounds is a cocktail comprising the following small molecules or chemical compounds: FGF8, SHH, CHIR99021, and WNT-C59. This combination of small molecules or chemical compounds is referred to herein as “FSCW cocktail” or “FSCW.”

In some embodiments, the FSCW cocktail further comprises B27, which is a serum-free nutritional supplement that promotes long term survival of in vitro cultured neurons, to form “FSCWB cocktail.” B27 supplement is available from various commercial vendors such as ThermoFisher. In some embodiments, FSCWB cocktail refers to a chemical cocktail comprising the following small molecules or chemical compounds: FGF8b, SHH, CHIR99021, WNT-C59, and B27. B27 can be contacted with DA NPCs at a final concentration in an in vitro culture of from about 1% to about 5% B27. As described in the Examples, DA NPCs cultured in the presence of the FSCWB cocktail can be expanded by 1000-fold over 6 passages without significant loss of DA identity while retaining the potential to generate functional DA neurons.

The term “cell culture medium” (also referred to herein as a “culture medium” or “medium” or “culture media”) as referred to herein is a medium for culturing cells containing nutrients that maintain cell viability and support proliferation. The cell culture medium can contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are available to those skilled in the art. Exemplary cell culture media that can be employed include mTESR-1 medium (StemCell Technologies, Inc., Vancouver, Calif.), or Essential 8 (E8) medium (Life Technologies, Inc.) on a MATRIGEL™ substrate (BD Biosciences, NJ) or on a Corning® Synthemax surface, or in Johansson and Wiles CDM supplemented with insulin, transferrin, lipids and polyvinyl alcohol (PVA) as substitute for Bovine Serum Albumin (BSA). Examples of commercially available media also include, but are not limited to, Dulbecco's Modified Eagle's Medium (DMEM), Minimal Essential Medium (MEM), Basal Medium Eagle (BME), knockout DMEM, Advanced DMEM/FI2, RPM1 1640, Ham's F-10, Ham's F-12, a-Minimal Essential Medium (aMEM), Glasgow's Minimal Essential Medium (G-MEM), Iscove's Modified Dulbecco's Medium, or a general purpose media modified for use with pluripotent cells, such as X-VIVO (Lonza).

In certain embodiments, it may be advantageous for the culture medium to further contain one or more supplements such as, for example, serum, knockout serum replacement (KSR), fetal bovine serum (FBS), Glutamax, non-essential amino acids, β-mercaptoethanol (β-ME), nucleosides, nucleotides, N2 supplement, Glutamax, bovine serum albumin (BSA), and combinations thereof “Supplemented,” as used herein, refers to a composition, e.g., a medium comprising a supplemented component, and not to the act of introducing the supplement to the medium.

In some embodiments, it is preferable to use a chemically defined culture medium. As used herein, the terms “chemically defined medium” and “chemically defined culture medium” are used interchangeably and refer to a culture medium containing formulations of fully disclosed or identifiable ingredients, the precise quantities of which are known or identifiable and can be controlled individually. As such, a culture medium is not chemically defined if (1) the chemical and structural identity of all medium ingredients is not known, (2) the medium contains unknown quantities of any ingredients, or (3) both. Standardizing culture conditions by using a chemically defined culture medium minimizes the potential for lot-to-lot or batch-to-batch variations in materials to which the cells are exposed during cell culture. Accordingly, the effects of various differentiation factors are more predictable when added to cells and tissues cultured under chemically defined conditions. As used herein, the term “serum-free” refers to cell culture materials that are free of or substantially free of serum obtained from animal (e.g., fetal bovine) blood. In general, culturing cells or tissues in the absence of animal-derived materials (i.e., under conditions free of xenogeneic material) reduces or eliminates the potential for cross-species viral or prion transmission.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

Obtaining Dopaminergic Neuron Progenitor Cells (DA NPCs)

The DA NPCs to be expanded can be obtained from a variety of sources. For example, the DA NPCs can be generated by differentiation of stem cells as described in U.S. Patent Publication 20140248696 (incorporated herein by reference in its entirety), which describes methods for generating populations of neuronal subtype-specific progenitors including midbrain dopaminergic neural progenitors by directed differentiation of neuroepithelial cells. The stem cells can be pluripotent stem cells, induced pluripotent stem cells, multipotent stem cells, unipotent stem cells, or combinations thereof. The method of differentiation induction of stem cells, including pluripotent cells, into a cell population comprising dopaminergic neuron progenitor cells is not restricted, and protocols are available and known to practitioners in the art.

As used herein, “pluripotent stem cells” appropriate for use according to a method of the invention are cells having the capacity to differentiate into cells of all three germ layers. Suitable pluripotent cells for use herein include human embryonic stem cells (hESCs) and human induced pluripotent stem (iPS) cells. As used herein, “embryonic stem cells” or “ESCs” mean a pluripotent cell or population of pluripotent cells derived from an inner cell mass of a blastocyst. See Thomson et al., Science 282:1145-1147 (1998). These cells express Oct-4, SSEA-3, SSEA-4, TRA-1-60 and TRA-1-81, and appear as compact colonies having a high nucleus to cytoplasm ratio and prominent nucleolus. ESCs are commercially available from sources such as WiCell Research Institute (Madison, Wis.). As used herein, “induced pluripotent stem cells” or “iPS cells” mean a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

Induced pluripotent stem cells exhibit morphological properties (e.g., round shape, large nucleoli and scant cytoplasm) and growth properties (e.g., doubling time of about seventeen to eighteen hours) akin to ESCs. In addition, iPS cells express pluripotent cell-specific markers (e.g., Oct-4, SSEA-3, SSEA-4, Tra-1-60 or Tra-1-81, but not SSEA-1). Induced pluripotent stem cells, however, are not immediately derived from embryos. As used herein, “not immediately derived from embryos” means that the starting cell type for producing iPS cells is a non-pluripotent cell, such as a multipotent cell or terminally differentiated cell, such as somatic cells obtained from a post-natal individual.

Subject-specific somatic cells for reprogramming into induced pluripotent stem cells can be obtained or isolated from a target tissue of interest by biopsy or other tissue sampling methods. In some embodiments, subject-specific cells are manipulated in vitro prior to use in a three-dimensional tissue construct of the invention. For example, subject-specific cells can be expanded, differentiated, genetically modified, contacted to polypeptides, nucleic acids, or other factors, cryo-preserved, or otherwise modified prior to differentiation into retinal progenitor cells according to the methods of this disclosure.

In some embodiments, the cells can be autologous or allogeneic cells (relative to a subject to be treated or who may receive the cells). Thus, somatic cells or adult stem cells can be obtained from a mammal suspected of having or developing a neurodegenerative condition or neuropathic disease, and the cells so obtained can be converted (reprogrammed) into DA NPCs that are expanded using the compositions and methods described herein.

In some embodiments, any of the above-referenced cells are cultured in a xeno-free cell culture medium (i.e., not comprising xenogenic materials). Of central importance for clinical therapies is the absence of xenogeneic materials in the derived cell populations, i.e., no non-human cells, cell fragments, sera, proteins, and the like. Culturing cells or tissues in the absence of animal-derived materials (i.e., under conditions free of xenogeneic material) reduces or eliminates the potential for cross-species viral or prion transmission.

Prior to culturing hPSCs (e.g., hESCs or hiPSCs) under conditions that promote differentiation into DA neuron progenitors, hPSCs can be cultured in the absence of a feeder layer (e.g., a fibroblast layer) on a substrate suitable for proliferation of hPSCs, e.g., MATRIGEL™, vitronectin, a vitronectin fragment, or a vitronectin peptide, or Synthemax®. In some embodiments, the hPSCs are passaged at least 1 time to at least about 5 times in the absence of a feeder layer. Suitable culture media for passaging and maintenance of hPSCs include, but are not limited to, mTeSR® and E8™ media. In some embodiments, the hPSCs are maintained and passaged under xeno-free conditions, where the cell culture medium is a chemically defined medium such as E8 or mTeSR, but the cells are maintained on a completely defined, xeno-free substrate such as human recombinant vitronectin protein or Synthemax® (or another type-of self-coating substrate). In some embodiments, the hPSCs are maintained and passaged in E8 medium on human recombinant vitronectin or a fragment thereof, a human recombinant vitronectin peptide, or a chemically defined self-coating substrate such as Synthemax®.

Any appropriate method can be used to detect expression of biological markers characteristic of cell types described herein. For example, the presence or absence of one or more biological markers can be detected using, for example, RNA sequencing, immunohistochemistry, polymerase chain reaction, qRT-PCR, or other technique that detects or measures gene expression. Suitable methods for evaluating the above-markers are well known in the art and include, e.g., qRT-PCR, RNA-sequencing, and the like for evaluating gene expression at the RNA level. Differentiated cell identity is also associated with downregulation of pluripotency markers such as NANOG and OCT4 (relative to human ES cells or induced pluripotent stem cells). Quantitative methods for evaluating expression of markers at the protein level in cell populations are also known in the art. For example, flow cytometry is typically used to determine the fraction of cells in a given cell population that express (or do not express) a protein marker of interest. In some embodiments, expanded DA neuron progenitor cell populations obtained by the methods of this disclosure comprise at least 80%, 85%, 90%, 95% and advantageously at least 98% DA NPCs that express biomarkers characteristic of DA NPCs: OTX2, FOXA2, SOX6, LMX1A, EN1, and CORIN.

Cell Therapy

In another aspect, provided herein are methods in which dopaminergic neuron progenitor cells expanded according to the methods of this disclosure are effectively used in the field of regenerative medicine for supplying dopaminergic cells which have been lost. Examples of diseases to which such cells can be applied include Parkinson's disease.

In some embodiments, DA neuron progenitor cells are isolated from a heterogeneous cell population, for example, by surface marker-based sorting (e.g., FACS), and the isolated DA neuron progenitor cells are expanded according to the methods of this disclosure, thus yielding a pure or substantially pure population having a sufficient number of DA neuron progenitor cells for cell therapy. As described in the Examples, DA neuron progenitors expanded by the methods of this disclosure retain the same DA neuron identity and possess the same therapeutic potency as the starting material when tested in the gold standard mouse model of Parkinson's disease. Advantageously, the methods also make it possible to expand progenitors for making DA neurons without batch-to-batch variations observed with conventional expansion methods.

In some embodiments, expanded dopaminergic neuron progenitor cells are differentiated into dopaminergic neurons (DAs) using any appropriate protocol such as the protocol for generating midbrain DA neurons described by Chen et al., Cell Stem Cell 18(6):817-826 (2016). In some embodiments, the Chen 2016 protocol is modified to use a medium comprising recombinant SHH (C25II, 100 ng/ml), FGF8b (100 ng/ml) and CHIR99021 (0.4 μM) for 4 days, starting on Day 9 of the protocol. After Day 14, the medium comprises recombinant SHH (C25II, 100 ng/ml), FGF8b (100 ng/ml), and the cells can be maintained in this medium for 1-2 weeks.

Drug Discovery Methods

In another aspect, this invention provides methods for producing and using an expanded population of DA neuron progenitor cells for high throughput screening of candidate test substances and identifying agents having therapeutic activity to slow, stop, and/or reverse progression of a neurodegenerative disease. Such agents may be used to treat neurodegenerative disease in subjects in need thereof. In some embodiments, an expanded population of DA neuron progenitor cells obtained as described herein can be screened to identify agents that modulate neural development and/or that cause neural toxicity.

In exemplary embodiments, the methods employ expanded populations of DA neuron progenitor cells obtained according to the methods of this disclosure for screening pharmaceutical agents, small molecule agents, or the like. For example, expanded populations of DA neuron progenitor cells are differentiated into DA neurons, and the DA neurons are contacted with a test substance. The contacted DA neurons can be studied to detect a change in a biological property of the neurons in response to exposure to the test substance.

Screening methods can comprise or consist essentially of (a) contacting a test agent to an expanded population of DA neuron progenitor cells or a population of DA neurons obtained by differentiating the expanded population of progenitors; and (b) detecting an effect of the agent on DA neurons or progenitors of the expanded population (e.g., disrupt or otherwise alter neural development, morphology, or function, or differentiation of neural cell types). In some embodiments, screening methods include screening candidate compounds to identify test agents that promote the development, morphology, and/or life span of human dopaminergic neurons. In some embodiments, candidate compounds can be screened for toxicity to human neural cell types or tissues. In some embodiments, detecting comprises detecting at least one positive or negative effect of the agent on morphology or life span of such cells and tissues, whereby an agent that increases or reduces the life span of human neural cell types or tissues, or has a positive or negative impact on the morphology of human neural cell types or tissues, is identified as having an effect on development of the human neural tube or neural tissues. In some embodiments, detecting comprises performing a method that is RNA sequencing, gene expression profiling, transcriptome analysis, cell proliferation assays, metabolome analysis, detecting reporter or sensor, protein expression profiling, Førster resonance energy transfer (FRET), metabolic profiling, and microdialysis. In some embodiments, the agent can be screened for an effect on gene expression, and detecting can comprise assaying for differential gene expression relative to an uncontacted biomimetic neural rosettes or cells derived therefrom.

In exemplary embodiments, detecting and/or measuring a positive or negative change in a level of expression of at least one gene following exposure (e.g., contacting) of a test compound to one or more biomimetic neural rosettes comprises whole transcriptome analysis using, for example, RNA sequencing. In such embodiments, gene expression is calculated using, for example, data processing software programs such as Light Cycle, RSEM (RNA-Seq by Expectation-Maximization), Excel, and Prism. See Stewart et al., PLoS Comput. Biol. 9:e1002936 (2013). Where appropriate, statistical comparisons can be made using ANOVA analyses, analysis of variance with Bonferroni correction, or two-tailed Student's t-test, where values are determined to be significant at P<0.05. Any appropriate method can be used to isolate RNA or protein from neural constructs. For example, total RNA can be isolated and reverse transcribed to obtain cDNA for sequencing.

As described herein, the methods of this disclosure are advantageous over conventional in vitro and in vivo methodologies for drug discovery screens. In particular, the methods described herein provide sensitive, reproducible, and quantifiable methods for screening test substances. It is possible rapidly screen test substances for therapeutic activity on pure populations of DA neurons with more reproducibility and predictability than screens using neurons obtained by other methods. Indeed, the in vitro screening methods can be conducted without the need for a human subject or animal models. These methods can be conducted economically (e.g., using multi-well plates that require minimal amounts of a test substance) and are readily adapted to high throughput methods (e.g., using robotic or other automated procedures). The methods are better alternatives to in vivo animal assays which are quantifiable assays but are error-prone, require a large number of animals, and are not easily standardized between laboratories or scalable for high-throughput screening. Shortcomings of animal-based assays have prompted regulatory agencies, including the Food and Drug Administration (FDA) and the United States Department of Agriculture, to promote the development of cell-based models comprising more physiologically relevant human cells and having the sensitivity and uniformity necessary for large-scale, quantitative in vitro modeling and screening applications (National Institutes of Health, 2008).

As used herein, “test substances” are not particularly limited and include, for example, single compounds such as natural compounds, organic compounds, inorganic compounds, proteins, antibodies, peptides, and amino acids, as well as compound libraries, expression products of gene libraries, cell extracts, cell culture supernatants, products of fermenting microorganisms, extracts of marine organisms, plant extracts, prokaryotic cell extracts, unicellular eukaryote extracts, and animal cell extracts. These may be purified products or crude purified products such as extracts of plants, animals, and microorganisms. Test compounds can include FDA-approved and non-FDA-approved drugs (including those that failed in late stage animal testing or in human clinical trials) having known or unknown toxicity profiles. Also, methods for producing test substances are not particularly limited; test substances may be isolated from natural materials, synthesized chemically or biochemically, or prepared by genetic engineering. “Test substances” also encompass mixtures of the above-mentioned substances.

Test compounds can be dissolved in a solvent such as, for example, dimethyl sulfoxide (DMSO) prior to contacting to an expanded population of DA neuron progenitor cells as described herein. In some embodiments, identifying agents comprises analyzing the contacted cells of the expanded population for positive or negative changes in biological activities including, without limitation, gene expression, protein expression, cell viability, and cell proliferation. For example, microarray methods can be used to analyze gene expression profiles prior to, during, or following contacting the plurality of test compounds to the expanded population. In some embodiments, a method of the present invention further comprises additional analyses such as metabolic assays and protein expression profiling.

Article of Manufacture

In another aspect, provided herein is a kit comprising one or more components useful for obtaining an expanded population of DA neuron progenitor cells. Components of the kit can include one or more compositions comprising small-molecules or chemical compounds that promote in vitro expansion of DA neuron progenitor cell, such as “FSCW” cocktail or “FSCWB” cocktail. The kit can also contain a chemically defined culture medium and one or more additional medium components or supplements. In some embodiments, the kit further comprises instructions for using expanded populations of DA neuron progenitor cells for screening test substances to identify those that exert a particular effect on DA neurons. In some embodiments, the kit further comprises instructions for differentiating expanded populations of DA neuron progenitor cells for use in cell therapies.

Unless defined otherwise, all technical and scientific terms used herein have the same meaning as commonly understood by one of ordinary skill in the art to which the invention pertains. Although any methods and materials similar to or equivalent to those described herein can be used in the practice or testing of the present invention, the preferred methods and materials are described herein.

In the specification and in the claims, the terms “including” and “comprising” are open-ended terms and should be interpreted to mean “including, but not limited to. . . . ” These terms encompass the more restrictive terms “consisting essentially of” and “consisting of.” As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising,” “including,” “characterized by,” and “having” can be used interchangeably.

As used herein, “a medium consisting essentially of” means a medium that contains the specified ingredients and those that do not materially affect its basic characteristics.

As used herein, “about” means within 5% of a stated concentration range, density, temperature, or time frame. Alternatively, and particularly in biological systems, the terms “about” and “approximately” may mean values that are within an order of magnitude, advantageously within 5-fold and more advantageously within 2-fold of a given value. Numerical quantities given herein are approximate unless stated otherwise, meaning that the term “about” or “approximately” can be inferred when not expressly stated.

The invention will be more fully understood upon consideration of the following non-limiting Examples.

### EXAMPLES

### Example 1—Small Molecule-Based Expansion of Dopaminergic Subtype Neuronal Progenitors from hPSCs Rescuing Parkinson's Disease Mice

This example illustrates the inventors' development and validation of a small-molecule based cocktail and methods for expanding human pluripotent stem cell (hPSC)-derived DA neural progenitor cells (DA NPCs) without loss of DA NPC identity or developmental potential. As described herein, DA NPCs in the presence of the small-molecule based cocktail retained their identity and developmental potency in vitro and following transplantation into a mouse model of Parkinson's disease. This strategy enables proliferation but maintains the fate identity of the progenitors, facilitating their application in high throughput assays and cell therapy.

Results

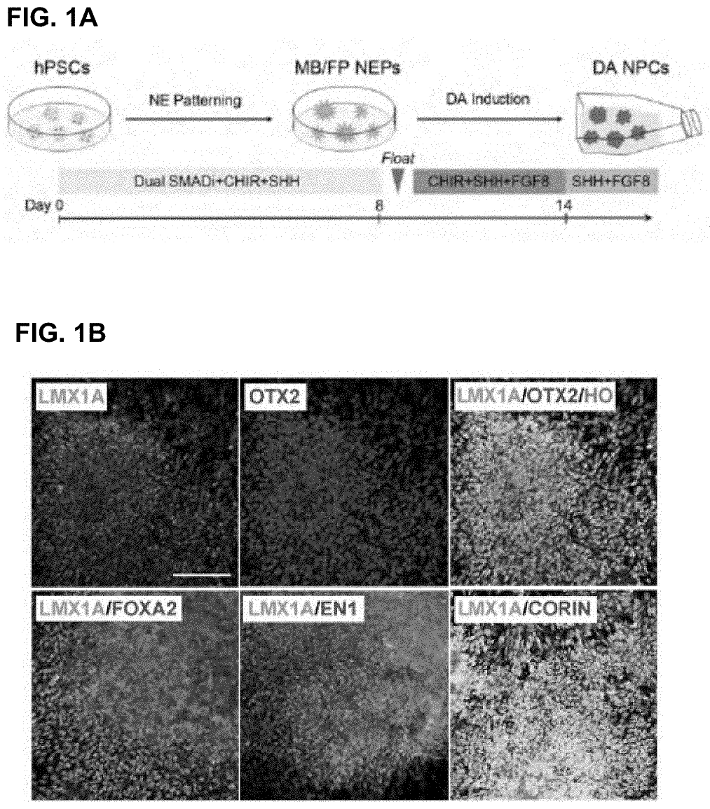

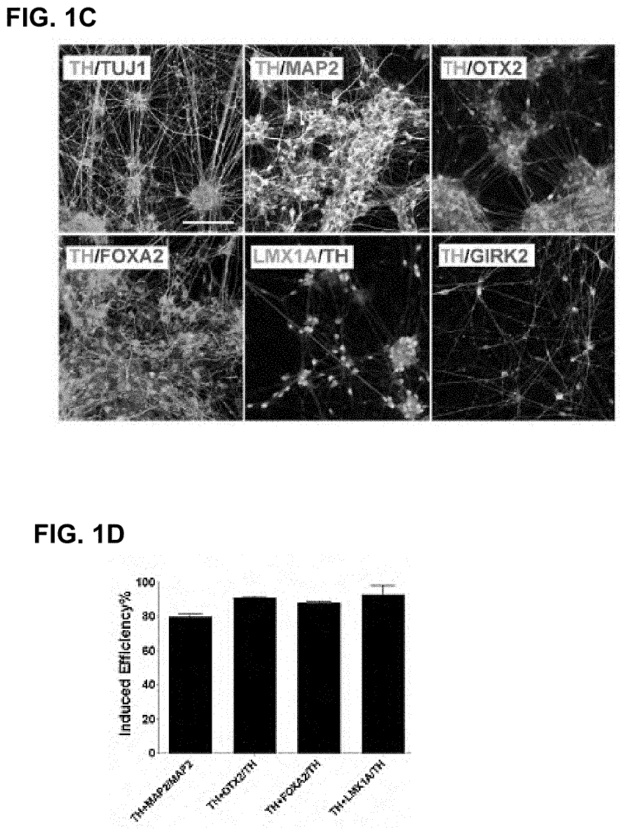

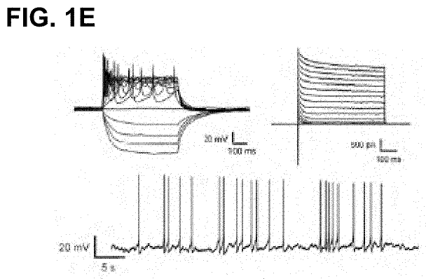

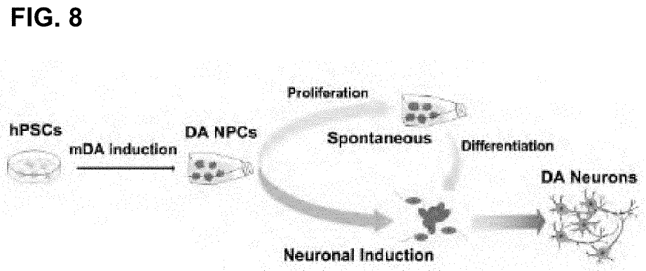

DA NPCs are efficiently induced from hPSCs: A protocol to differentiate hPSCs to DA neurons efficiently was previously developed (Chen et al., 2016; Xi et al., 2012) (FIG. 1A). Under these conditions, DA NPCs appeared in 3-4 weeks of hPSC differentiation, as evidenced by co-immunostaining of DA signature transcriptional factors (LMX1A/OTX2, LMX1A/FOA2, and LMX1A/CORIN/EN1>80%; LMX1A/EN1>50%) (FIG. 1B). Following two weeks of maturation (FIG. 8) (Chen et al., 2016), the DA NPCs developed extensive neurites and co-expressed mature DA neuronal markers, including TUJ1, MAP2, and tyrosine hydroxylase (TH) (FIGS. 1C-1D). Electrophysiological recording showed functional maturation of the differentiated neurons, as indicated by typical Na⁺/K⁺ currents, action potentials, and spontaneous firing pattern at a low frequency (FIG. 1E).

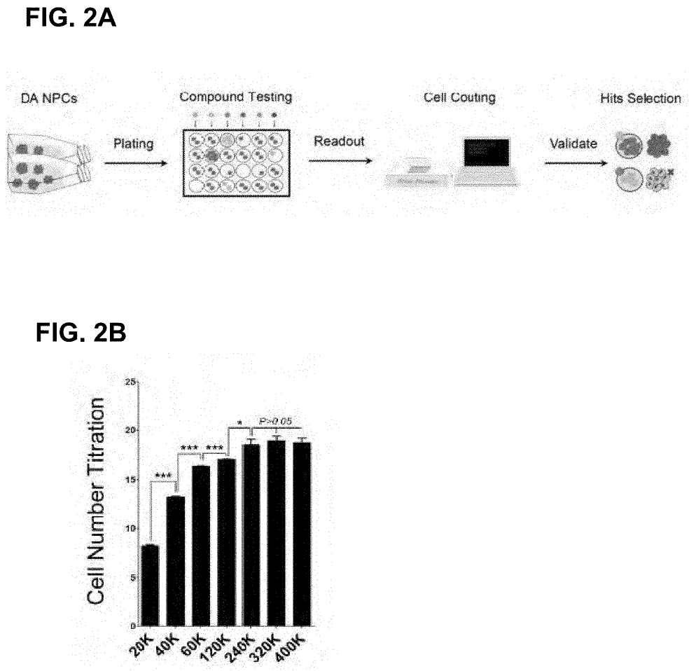

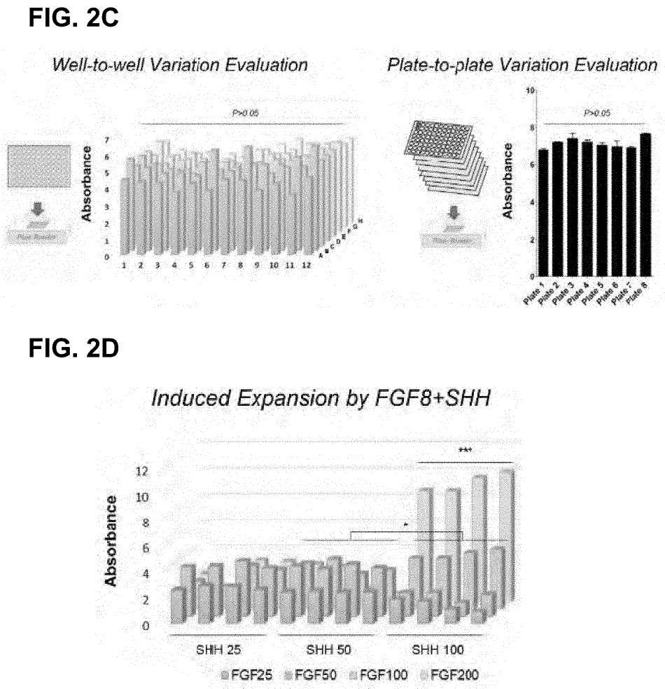

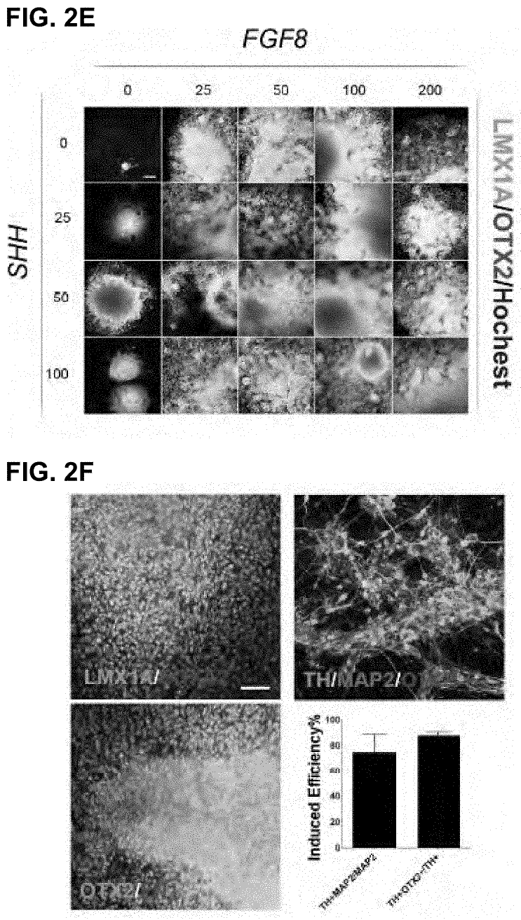

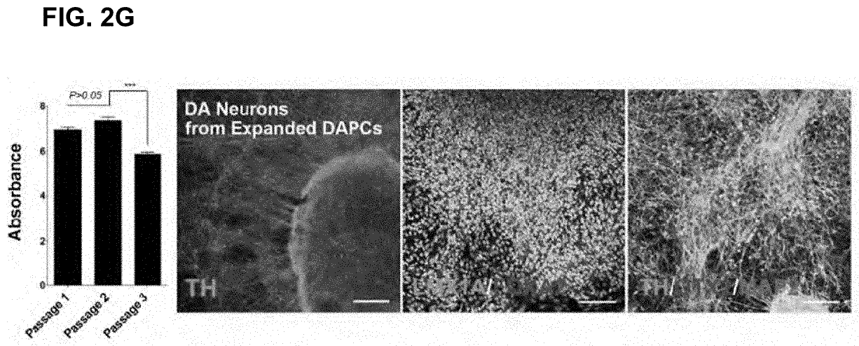

FGF8 and SHH are not sufficient for expanding DA NPCs: To determine if and how the DA NPCs can be expanded, a CKK8-based assay for quantifying cell numbers was first established. This assay was based on the absorbance readout from a plate reader to estimate the total cell number by monitoring mitochondrial activity, which was used for primary screening/testing for candidate molecules and validating the hits (FIG. 2A).

The optimal seeding density was first determined by plating increasing numbers of DA NPCs to each well. The CKK8 absorbance exhibited a linear relationship with the increased cell number, between 20,000 and 120,000 cells/well. The absorbance reached a plateau when the seeding density was beyond 120,000 cell/well (FIG. 2B), and 20,000 cells/well were used for subsequent assays. Using this optimal seeding density (20,000/well), well-to-well and plate-to-plate variation (FIG. 2C) was examined. There were no obvious well-to-well and plate-to-plate variations (FIG. 2C). Hence, this assay is consistent under this seeding density.

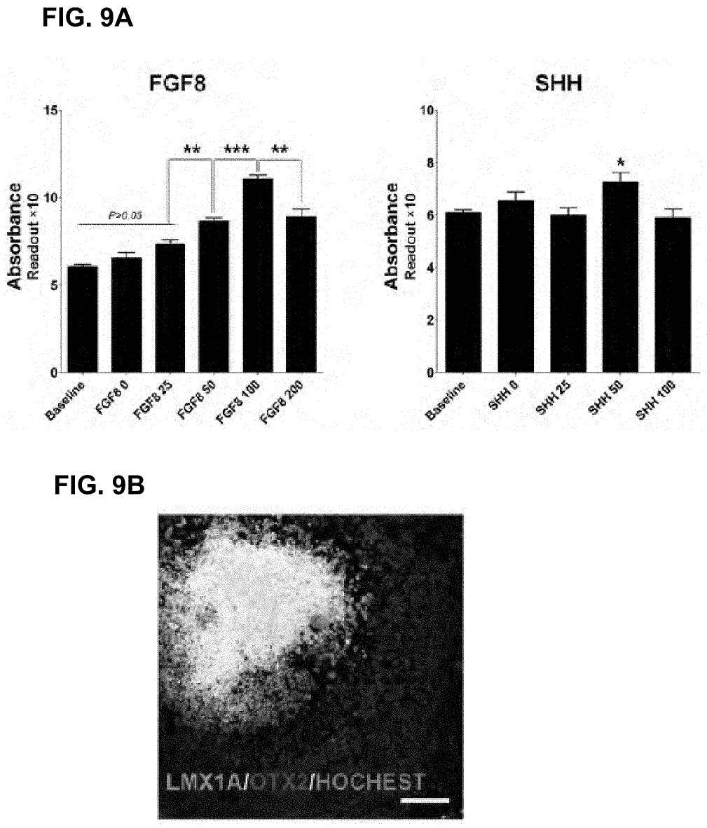

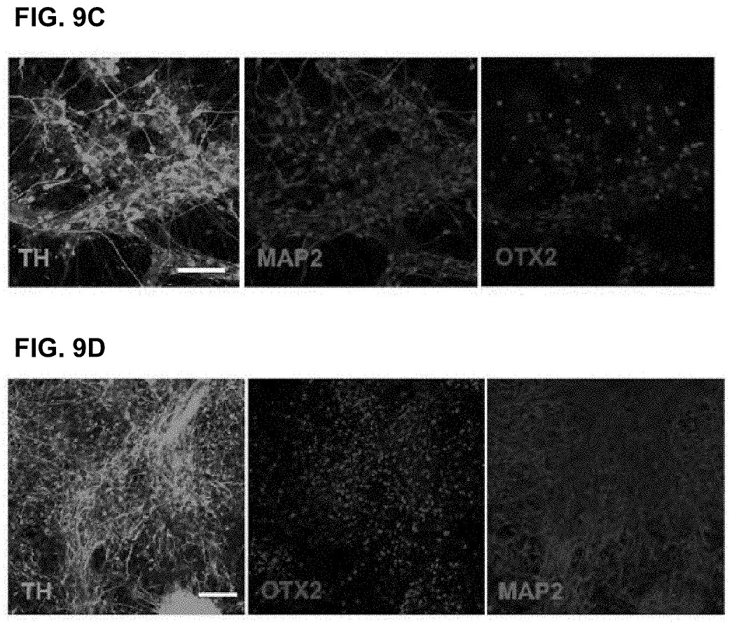

FGF8 and SHH are critical to induce the identity of the DA NPCs (Chen et al., 2016; Xi et al., 2012). The capacity for FGF8 and SHH to expand DA NPCs while maintaining the DA identity was thus determined. By using different dose combinations (SHH at 25, 50, and 100 ng/ml plus FGF8 at 25, 50, 100 and 200 ng/ml), at a higher dose of FGF8 (100 or 200 ng/ml), the DA NPCs increased in numbers significantly (FIG. 2D). However, the expanded NPCs lose the DA NPC identity, as shown by the loss of co-expression of LMX1A and OTX2 (FIG. 2E). At the dose of 50 ng/ml for FGF8 and 25 ng/ml for SHH, the DA NPCs were expanded modestly for one passage at the ratio of 1:3, in a 5 day expansion without losing the co-expression of LMX1A and OTX2 (FIGS. 2D-2F; FIGS. 9A-9B). However, under this combination of FGF and SHH, the proliferative rate of DA NPCs decreased when further expanded by passage 3 even though the DA identity was retained (FIG. 2G; FIGS. 9C-9E).

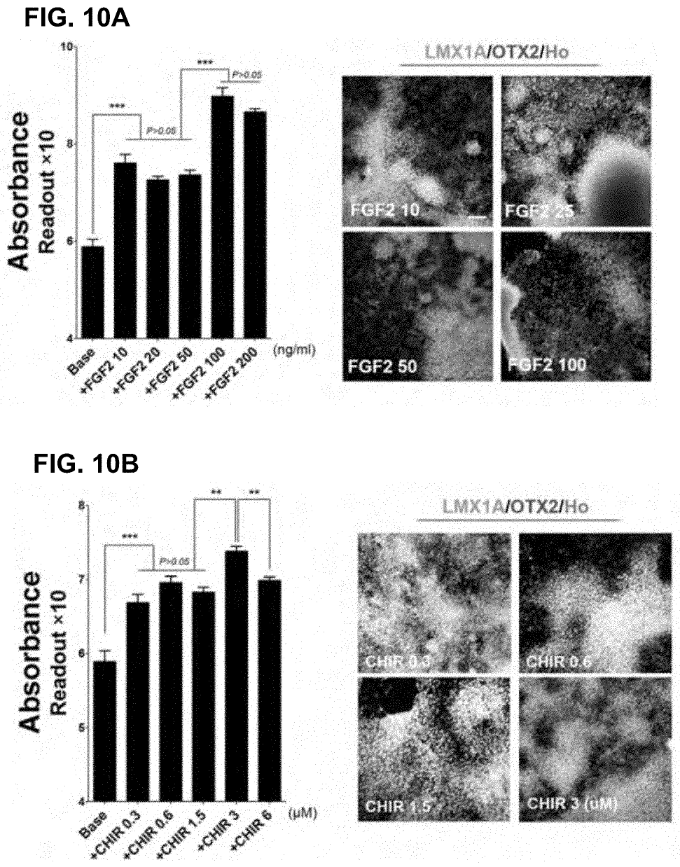

FGF2 and CHIR99021 have been reported for expanding pan-NPCs or specified NPCs (Du et al., 2015; Taupin et al., 2000). FGF2 alone, at a dose from 10 to 200 ng/ml, induced dramatic cell proliferation, but resulted in the loss of co-expression of LMX1A and OTX2 in one passage in 5 days (FIG. 10A). Similarly, CHIR alone expanded the DA NPCs in a range of doses but the DA NPC identity (co-expression of LMX1A and OTX2) was retained only in a narrow dose window (0.6 μM). Lower or higher CHIR concentrations resulted in the loss of DA identity (FIG. 10B), which is consistent with previous reports that CHIR patterned neural cell identity along the anterior-posterior (AP) and dorsal-ventral (DV) axis (Tao and Zhang, 2016). Thus, new components are required for further expanding the DA NPCs.

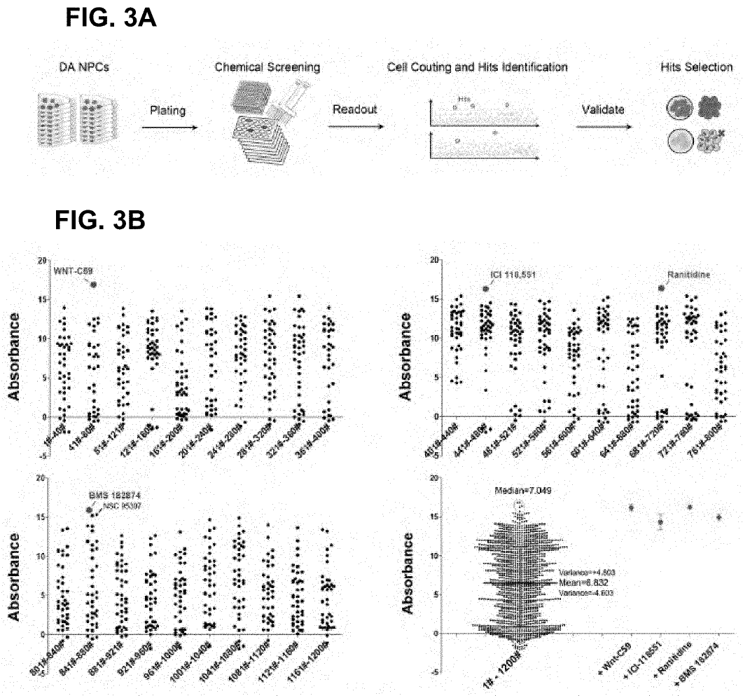

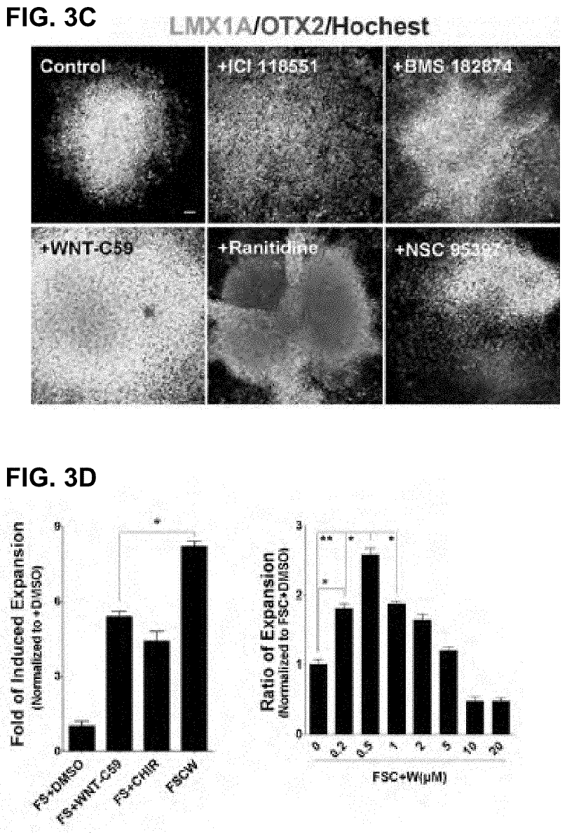

Chemical screening identifies an additional compound that expands DA NPCs: The chemical screening platform was set up using FGF8+SHH as a basal condition, FGF8+SHH+DMSO as a control, and FGF8+SHH+1 as the screening mode. With the standardized screening platform (FIG. 3A), 1,200 small molecules (Tocris Total) were screened and the top 5 candidate compounds that increased the absorbance by >2 folds over the median absorbance (FIG. 3B) were identified. These candidates were validated by manual counting the cell number after expansion (1 passage; 5 days; ration 1:3) and it was found that only one hit from the five, WNT-C59 as a WNT antagonist, maintained DA identity after induced expansion, evidenced by the co-expression of LMX1A and OTX2 (FIG. 3C). At 2 WNT-C59 induced a 5-fold expansion on top of FGF8+SHH (FIG. 3D). We referred to it as the “FSW” cocktail.

DA NPCs are expanded by an optimized cocktail: Because WNT-C59 is a WNT antagonist and CHIR at a low dose (0.6 μM) expanded the DA NPCs but did not alter their cellular identity (FIG. 10B), addition of CHIR to the FSW cocktail (yielding “FSCW” cocktail) was proposed to yield additive effect. When tested, a further 50% increase in cell number was observed, compared to using FSW (FIG. 3D). Because of the opposing effects of WNT-C59 and CHIR (i. e. in terms of their effects on the WNT pathway), the dose of WNT-C59 was optimized by titration starting from 0 μM, until that causing cell death (observed at 20 μM) (FIG. 3D). WNT-C59 exerted the maximum effect in this cocktail at the concentration of 0.5 μM (FIG. 3D). The small molecule cocktail was further optimized by adjusting medium supplements. The addition of B27 (yielding “FSCWB” cocktail) further increased the cell number by 3-fold (FIG. 3E). Using the optimized “FSCWB” cocktail, DA NPCs were expanded starting from 1 million in one well of a 6-well plate and by passaging the cells when the cell population reached around 70% to 90% confluence at 1:3 ratio every 5 days. By 6 passages, the DA NPCs were expanded by about 1000 times in the presence of the cocktail (FIG. 3F).

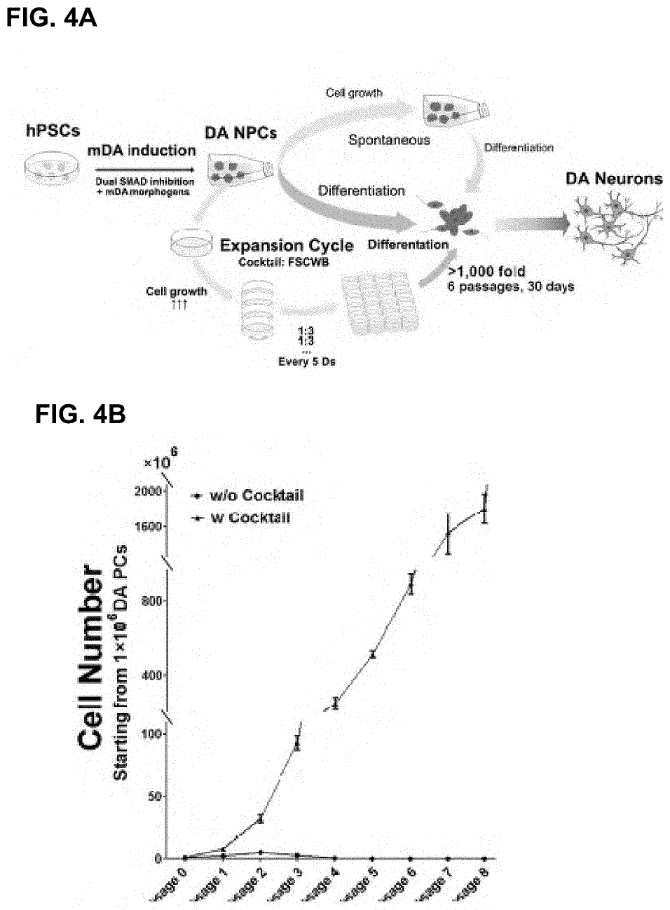

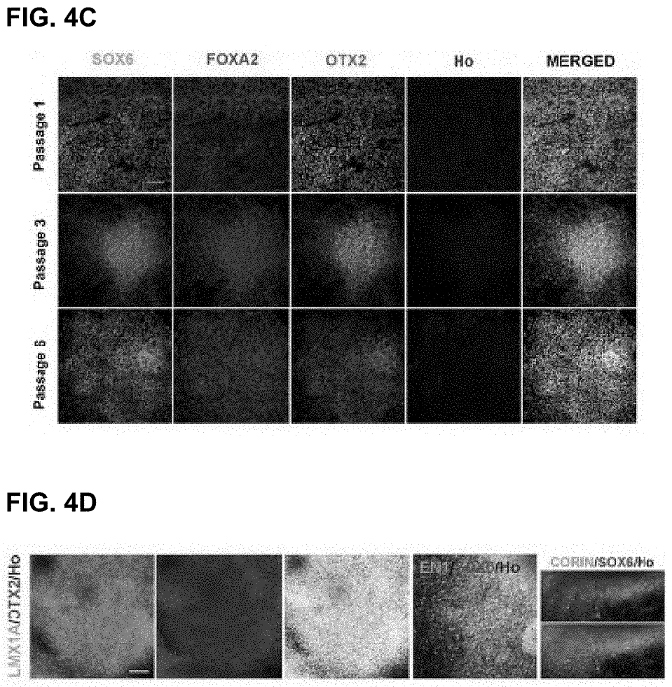

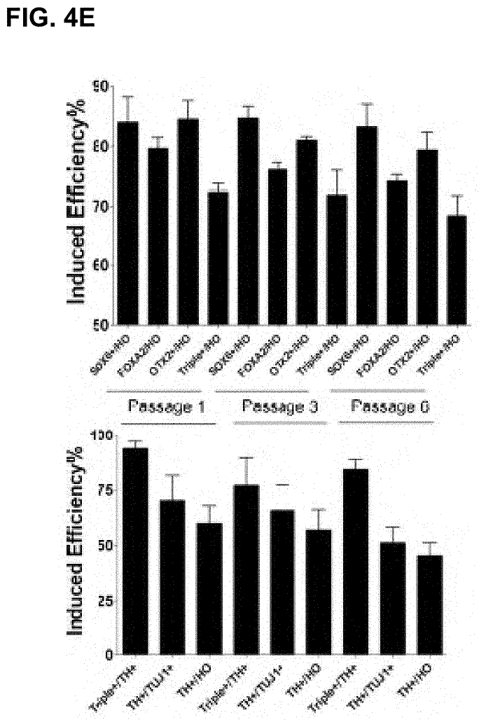

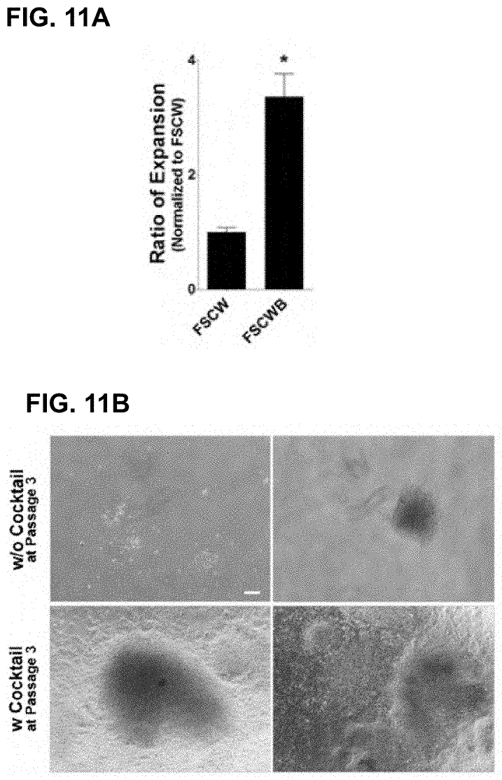

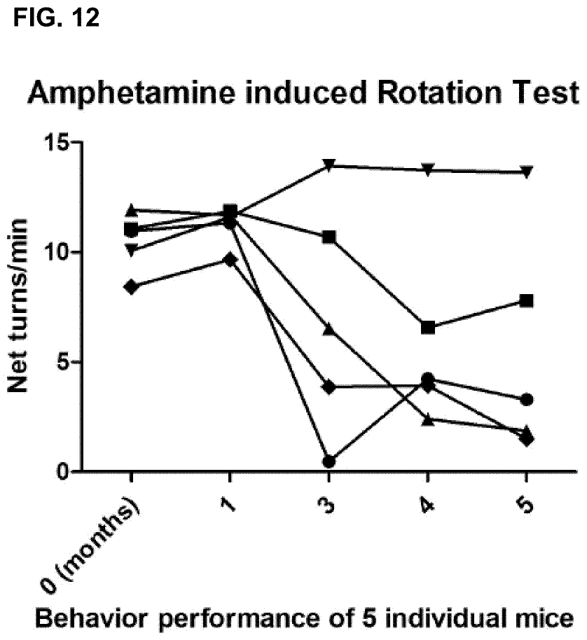

Expanded DA NPCs retain the cell identity and differentiation potency: To determine if the expanded cells retain the identity of DA NPCs, the cells were immunostained for their expression of FOXA2 and OTX2 at passages 1, 3, 6, and 8. In addition, the cells were stained for SOX6, a critical determinant for the development of midbrain DA neurons by coordinating with OTX2 to define the subpopulation of substantial nigra DA neurons at the neural progenitor stage Panman L et al., Cell Rep. 8(4): 1018-25 (2014). As indicated by the quantification of SOX6/FOXA2/OTX2 single- and triple-positive cells, a large proportion of the cells expressed these marker (triple-positive: 72.2%, 71.8%, and 68.4% for passage 1, 3, and 6, respectively) (FIGS. 4C and 4E; FIG. 11B), suggesting their maintenance of the DA NPC identity. Further expansion resulted in progressive reduction in the triple positive cell proportion, reaching less than 50% by passage 8. NPCs were therefore expanded for 6 passages using our FSCWB cocktail, which enabled 1000-fold expansion. The DA identity of the expanded progenitors at passage 6 was further confirmed by their co-expression of other hallmark DA genes, including LMX1A, EN1, and CORIN in addition to SOX6, FOXA2, and OTX2 (FIG. 4D).

To determine if the expanded DA NPCs retain the differentiation potential, NPCs were differentiated from passage 1, 3, and 6 after expanding for 5 days/passage. Quantitative analysis indicated that >50% of the cells expressed TH at passage 1, 3, and 6 (FIG. 4E), indicating that they are DA neurons.

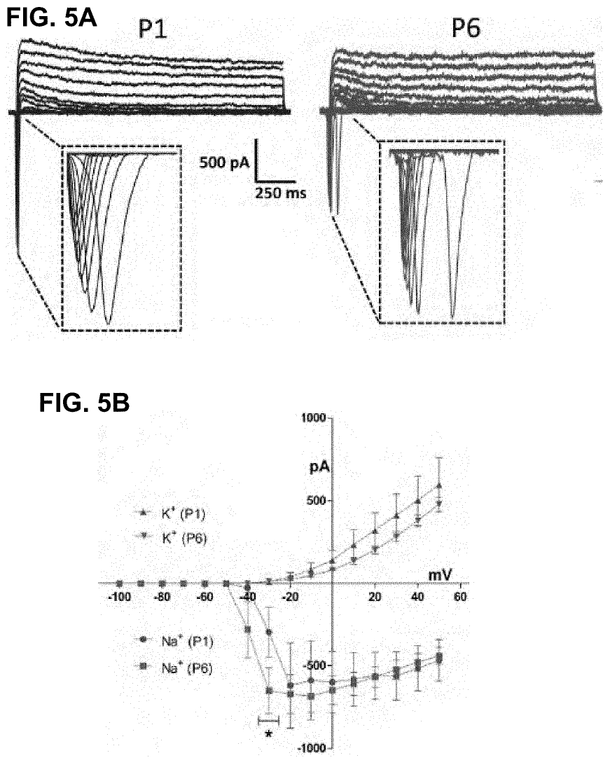

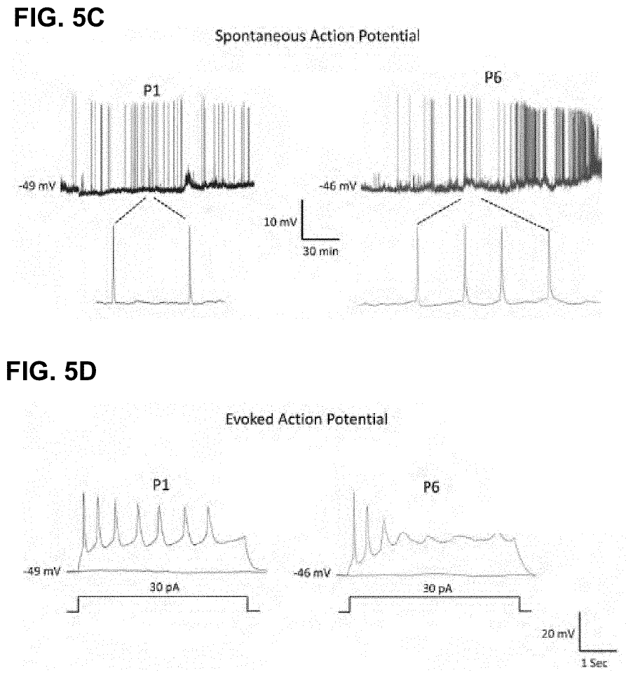

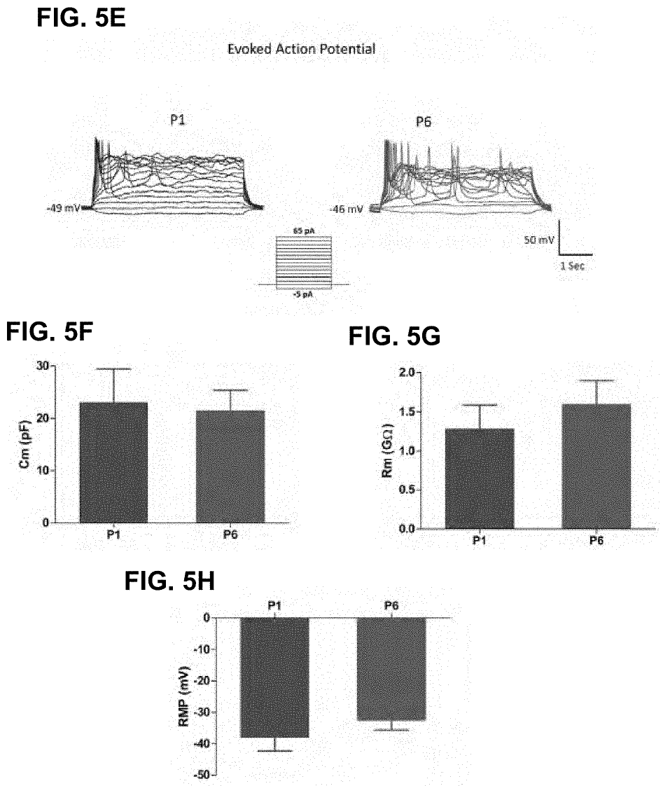

Whole cell recordings were performed to analyze functional activities of the dopaminergic neurons derived from passage 1 and passage 6. Both passages exhibited voltage-gated inward and outward currents when stimulated by depolarizing voltage steps (FIG. 5A). Analysis of passive membrane properties did not show any significant differences between P1 and P6 except when the voltage=−30 mV (FIG. 5B). Similarly, both P1 and P6 derived neurons showed spontaneous firing of action potentials (FIG. 5C). Evoked action potentials were observed when neurons were stimulated by current steps in both passages (FIGS. 5D and 5E). There is no statistical difference between passage 1 and passage 6 DA neurons for the spontaneous action potential and evoked action potential (FIGS. 5F-5H).

Taking together, DA NPCs were expanded by 1000-fold over 6 passages without significantly losing the DA identity and retaining the potential to generate functional DA neurons (FIG. 4A).

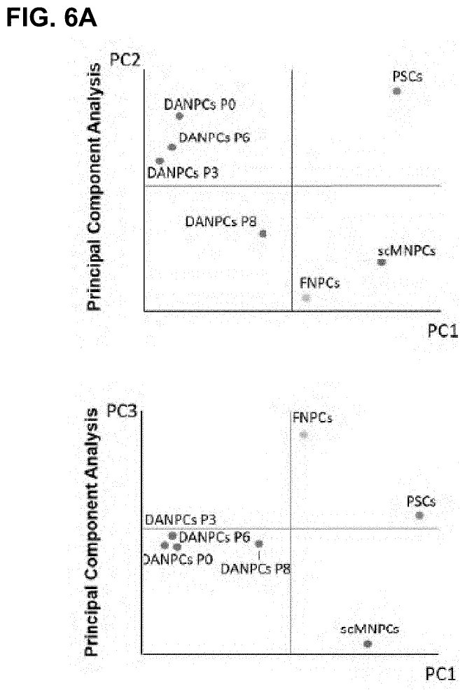

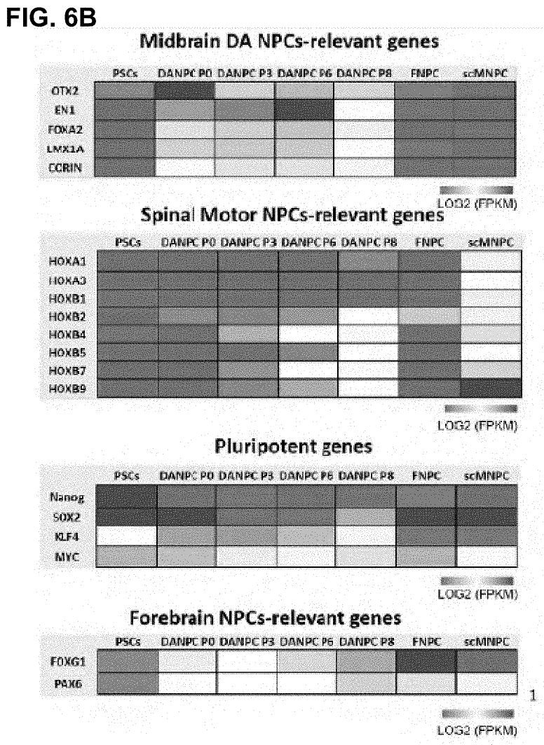

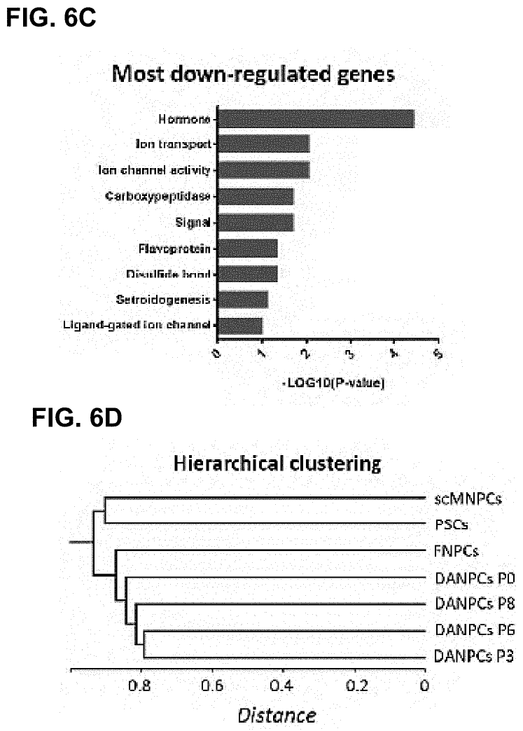

Global gene expression analysis confirms the identity of DA NPCs: Expansion of regionally specified neural progenitors often results in the change of positional identity due to the effect of mitogens. RNAseq analysis was performed on the DA NPCs across passage 1, 3, 6, and 8, along with undifferentiated PSCs, the forebrain NPCs and the spinal cord NPCs, generated from the same PSCs according to our published protocols (Li et al., 2009, Du et al., 2015), as controls. Principal component analysis (PCA) showed that these DA NPCs retained their distinct DEG from the other region-specific NPCs (forebrain NPCs and spinal NPCs). Principal Component Analysis (PCA) was performed on generated DA NPCs at different passages. DA NPCs were distinguishable from the not-induced pluripotent stem cells or other subtype neuronal progenitors (FIG. 6A). By looking into depth, DA NPCs at passage 1, 3 and 6 grouped closer than DA NPCs at passage 8 under different subset of component grouping analysis (FIG. 6A), indicating that the gene expression pattern of DA NPCs is maintained for at least 6 passages.

Analysis of DEG revealed that the DA NPCs expressed a similar level of DA related genes, including OTX2, EN1, FOXA2, LMX1A and CORIN over 6 passages whereas the expression intensity of EN1, LMX1A and CORIN decreased at passage 8 (FIG. 6B). This change in gene expression pattern is consistent with the observation made at the cellular level. There was no expression of pluripotent, forebrain, or spinal cord genes within the 6 passages of DA NPC expansion, further confirming the maintenance of midbrain (DA) identity during the expansion (FIG. 6B).

Comparison of gene expression at passage 8 with passage 6 revealed that the most up-regulated genes were grouped to HOX family-related genes (FIG. 6C), suggesting a shift of the regional identity to a more caudal fate with extended expansion. The most down-regulated genes enriched around the gene classes of hormone, ion transport, ion channel activity and others (FIG. 6C). Hierarchical clustering indicated that the expanded DA NPCs clustered closely together which are identical to each other in terms of the global expression pattern as reflected by the distance of correlation and were distinct from undifferentiated PSCs, forebrain NPCs and spinal NPCs (FIG. 6D). These results provide a foundation for manipulation of related pathways for further expansion of the DA NPCs.

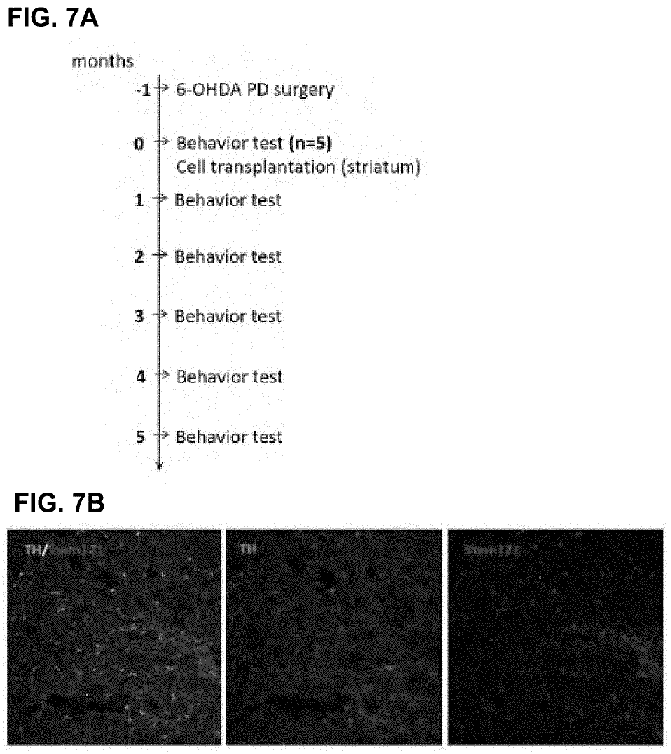

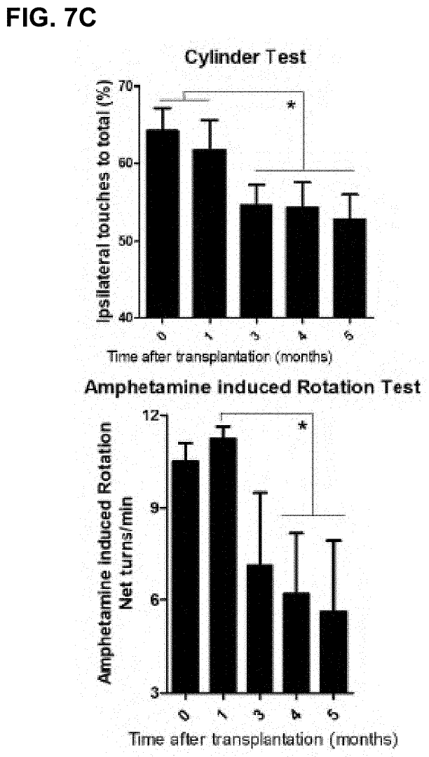

Transplantation of expanded DA NPCs rescues motor deficits of PD mice: To determine if expanded DA progenitors retain the therapeutic potential like those un-expanded, expanded DA progenitors at passage 6 were transplanted into the striatum of adult SCID mice lesioned by injection of 6-hydroxydopamine (6-OHDA) (also known as Oxidopamine or 2,4,5-trihydroxyphenethylamine) into the substantial nigra, as previously described (FIG. 7A) (Chen et al., 2016). 6-OHDA is a neurotoxic synthetic organic compound widely used to induce the main cellular processes involved in Parkinson's disease (PD), such as oxidative stress, neurodegeneration, neuroinflammation, and neuronal death by apoptosis.

Behavioral analysis indicated that the PD mice receiving medium injection (control) did not show changes in cylinder test and amphetamine-induced rotation test at 1, 3, 4, and 5 months post transplantation. Histological examination at five months showed the presence of grafts in the striatum of transplanted brains, as indicated by hNu+ cells. >80% of the human-specific fibers (labeled by STEM121) are co-labeled with TH, the marker of dopaminergic neurons (FIG. 7B). Those receiving the transplantation with expanded DA NPCs showed a reduction in unilateral forelimb touching, revealed by cylinder test and amphetamine-induced rotation from 3 months post-transplantation (FIG. 7C). These results suggest that the DA progenitors, after expansion in the FSCWB cocktail and transplantation into the striatum, retained the capacity to restore the motor function in the PD mice.

Significance

Disclosed herein is a chemical cocktail for expanding DA NPCs by 1000-fold. The expanded DA NPCs retained their identity by maintaining the midbrain floor plate character and the gene expression profile of DA NPCs. These cells exhibited a similar capacity to differentiate into DA neurons in vitro and in vivo as their unexpanded cells, contributing to the restoration of motor function in a PD mouse model. The ability to expand the lineage-committed NPCs enabled production of large quantities of specialized NPCs with a consistent quality, facilitating their application in drug discovery and cell therapy.

Expansion of neural progenitors is typically achieved by growing the cells in the presence of FGF2 and/or EGF. This approach, however, resulted in loss of regional identity of the progenitors, hence the fate of the expanded cells. The identity of spinal motor neuron progenitors had been shown to be maintained during proliferation by tightly regulating the dorsal-ventral identity of the progenitors using a small molecule cocktail (Du et al., 2015). There are also reports expanding the progenitors of functional non-neural cell types. In particular, it has been shown to be feasible to expand the pancreatic progenitors and hepatocytes progenitors by either co-culturing with organ-matched mesenchyme or chemically-induced dedifferentiation (Sneddon et al., 2012; Fu et al., 2019). For maintenance of midbrain DA progenitors, it is essential to maintain the ventral midbrain identity of the progenitors. Interestingly, the critical molecule identified herein was a WNT antagonist. Without being bound by mechanism the DA NPCs tend to become caudalized (hindbrain) when expanded in the presence of CHIR, and WNT-C59 neutralized the caudalizing effect of CHIR, thus balancing rostro-caudal identity of the progenitors. Of course, WNT-C59 may also regulate the dorsal-ventral identity of the progenitors. This indicated a need to adjust the concentration of SHH. By doing so, the fate of the DA NPCs can be maintained during proliferation for a period of time.

By the methods disclosed herein, continual self-renew of the DA NPCs was not achieved. However, the degree of expansion bears significant implication value. Beginning with one million progenitors, one billion cells can be produced after 1000-fold expansion using the disclosed methods. This can produce sufficient number of cells of the consistent quality for HTS or cell therapy. This is particularly useful for cell types that presently do not differentiate efficiently. In addition, the observed increase of non-neural and non-DA neuronal cells in the population with extended culture for expansion raises a further demand for possibly developing a chemical cocktail exclusively expanding DA neuronal progenitors. In this embodiments, progenitors can be isolated (e.g., by surface marker-based sorting) and expanded as described herein, thus producing a sufficient number of target cells. On the other hand, these findings indicate signaling pathways underlying the self-renew of subtype-specific neuronal progenitors, such as fine-tuning the WNT signaling pathway by using WNT agonist and antagonist in a balanced manner towards maintaining a specified subtype cell fate. These findings enable methods for realizing the self-renew of subtype neuronal progenitors as an ultimate end by probing defined self-renewing relevant signaling pathways.

In total, these findings demonstrate the feasibility of expanding a rare subtype-specified neuronal population in culture dish by small-molecule based chemical approach, in an enriched manner. Regarding the study for expanding DA subtype neuronal progenitors as an example, the strategy and principles developed herein for expansion of committed progenitors using small molecule-based chemical approach could be applicable for other subtype neuronal progenitors, other tissue types or cell lineages.

Methods

HPSC culture: HESCs (line H9, passages 20-40) were cultured as previously described (Chen et al., 2016). Briefly, cells were passaged weekly by using Dispase (1 mg/ml, Gibco) and plated on a layer of irradiated mouse embryonic fibroblasts (MEFs). The hPSC culture medium consisted of DMEM/F12 basal medium, 20% Knockout serum replacement (KSR), 0.1 mM β-mercaptoethanol, 1 mM 1-glutamine, non-essential amino acids (Gibco) and 4 ng/ml FGF-2 (R&D Systems).

Generation and expansion of DA NPCs: DA NPCs were generated as previously described, in particular FGF8b was added from Day 9 (Chen et al., 2016; Xi et al., 2012) (FIG. 1A). The generated DA NPCs were passaged at 10⁶ cells/well on a MATRIGEL™ (1:30; PBS diluted and store in 4° C. for use in 2 weeks) coated 6-well plate as the first passage. After overnight, incubation, the medium was switched to the FSCW cocktail in the expansion medium. FSCW cocktail contained FGF8b (50 ng/ml; Peprotech), SHH (25 ng/ml; Peprotech), CHIR (0.6 μM; Tocris) and WNT-C59 (0.5 μM: Tocris). The expansion medium consisted of DF12 basal medium (ThermoFisher), B27 (100×; ThermoFisher), Glutamax (100×; ThermoFisher) and NEAA (100×; ThermoFisher). The cells were passaged every 5 days at the ratio of 1:3. To digest the cells, the cultures were incubated in PBS-diluted EDTA (1:100; Thermo) at 37° C. for 5 minutes before lifting the cells by adding 2 ml DF12 basal medium using cell lifter (Coring) and collecting the cells into a centrifuge tube. Following centrifugation at 1000 rpm for 2 minutes and removal of the supernatant, the cells were resuspended in the expansion medium with FSCW cocktail and seeded onto the coated plates as described above. Y27632 (10 uM; Tocris) was added overnight to improve cell survival.

Immunostaining and Quantification: Cells on coverslips/wells were rinsed with PBS and fixed in 4% paraformaldehyde for 20 minutes. After rinsing with PBS twice, cells were treated with 0.3% Triton for 10 min followed by 10% donkey serum for 1 hour before incubating with primary antibodies overnight at 4° C. Cells were then incubated for 1 hour at room temperature with fluorescently-conjugated secondary antibodies (Life Technologies). The nuclei were stained with Hoechst (Ho) (Sigma-Aldrich). Images were taken with a Nikon A1R-Si laser-scanning confocal microscope (Nikon, Tokyo, Japan). The primary antibodies used include the followings: Goat anti-OTX2 (1:1000, R&D), Rabbit anti-LMX1A (1:500, Abcam), Goat anti-OTX2 (1:500, R&D systems), Goat anti-FOXA2 (1:500, Santa Cruz), Mouse anti-EN1 (1:200, 4G11-C, DSHB), Rat anti-Corin (1:100, R&D Systems), Rabbit anti-TH (1:500, Pel-Freez Biologicals), Mouse anti-TUJ1 (1:500, Santa Cruz Biotechnology), Mouse anti-MAP2 (1:200, Sigma-Aldrich), Rabbit anti-GIRK2 (1:80, Alomone Labs), Rabbit anti-SOX6 (1:1000, Sigma-Aldrich), Mouse anti-Stem121 (1:500, Clonetech). For quantifying DA NPC populations, cells were counted among the total Hoechst labeled nuclei. Three independent cultures (n=3) were analyzed.

Quantitative reverse transcription polymerase chain reaction (qRT-PCR): RNA was extracted by the Qiagen RNeasy kit and quantified. 500 ng of RNA was used for reverse transcription using Bio-Rad iScript (1708891). iTaq universal SYBR Green Supermix (1725124) was used for qPCR reactions. Values were normalized to GAPDH.

Electrophysiology: Whole-cell patch-clamp recordings were performed on neurons seeded on glass coverslips. Data acquisition was made using Multiclamp 700B amplifier and pClamp 11.0 software (Molecular Devices, Palo Alto, Calif.). Offline analysis was performed using Clampfit 11.0. For all experiments, a series resistance of up to 25 MS2 was tolerated. All reagents for patch clamp experiments were purchased from Sigma. Voltage clamp and Current clamp recordings were measured in artificial cerebrospinal fluid (ACSF) containing (mM): 148 NaCl, 4.2 KCl, 5 Glucose, 5 HEPES, 1 CaCl2, 0.5 MgCl2, pH 7.4 with NaOH, 310-320 mOsm. Patch glass pipettes with OD 1.5 mm×ID 0.86 mm (Sutter instruments) were pulled with P-97 Sutter pipette puller (Sutter Instruments, CA). The recording electrode β-6 MS2) was filled with internal solution containing (mM): 130 K-Gluconate, 6 KCl, 3 NaCl, 0.5 MgCl2, 5 HEPES, 2 EGTA, 1 Mg-ATP, 0.5 Na-GTP, 1 Sodium phosphocreatine, pH 7.3 with KOH, 280-290 mOsm. Cells were clamped at −70 mV for neuronal current measurements. Under voltage clamp mode, neuronal currents were evoked by injecting steps of 250 ms depolarizing voltages starting from −100 to +60 mV with 10 mV increment. For current clamp experiments cells were held at 0 pA. In current clamp mode, induced action potentials were evoked by injecting a series of 1 sec current steps starting from −5 pA to 65 pA with an increment of 5 pA. Spontaneous neuronal firing was assessed for 30 minutes.

Generation of Parkinson's disease mice: The Parkinson's disease modeling was performed as previously described (Chen et al., 2016). Briefly, the SCID mice (12 weeks of age) were anesthetized by O₂-vaporized Isoflurane gas. The heads of animals were then fixed in a stereotactic frame (Kopf instruments) and 1 μl 6-OHDA (3 mg/ml) was slowly injected into the Substantial Nigra (coordinates A.P.=−2.9 mm. M.L.=+1.1 mm, D.V.=−4.5 mm) via microinjection pump (Steolting). One month after PD modeling, all the animals were subjected to the behavioral tests to evaluate their motor behaviors and those which show over 6 rotations per minute in the amphetamine induced rotation test were used for cell transplantation.

DA NPC transplantation: The DA NPCs at passage 6 were digested by Accutase (Innovative Cell Technologies) and resuspended (200,000 cells in 2 μl/mice) in the artificial cerebrospinal fluid aCSF with BDNF (20 ng/ml), B27 (1:50), and ROCKi (Chen et al., 2016). Animals were anesthetized by isoflurane (1%-2%) inhalation. Under stereotaxic guidance about 2×10⁵ cells were slowly injected into the ipsilateral striatum (coordinates A.P.=+0.6 mm, M.L.=+1.8 mm, D.V.=−3.2 mm) manually.

Amphetamine induced rotation test: Amphetamine-induced rotations were done as previously described (Chen et al., 2016). The animals were administrated with 5 mg/kg amphetamine (5 μl/g in 1 mg/ml concentration, i.p., Sigma Aldrich) and placed into the rotation chamber ten minutes after injection. The rotation behavior was recorded by the video camera for 90 min and analyzed by the investigators blind to the subjects ID. Data were presented as the average ipsilateral net rotations per minute.

Cylinder test: The subjects were placed in an acrylic cylinder and their movements were recorded by the video camera for 3 min. The numbers of the ipsilateral and contralateral forelimb touch onto the wall of the cylinder were counted. Data were shown as the percentage of the ipsilateral touches to the total touches (ipsilateral+contralateral). Minimum touch number is 20.

Immunohistochemistry on brain slices: The animals were sacrificed at the indicated time points after transplantation with an overdose of pentobarbital (250 mg/kg, i.p.) and perfused intracardiacally with 40 ml normal saline, followed by 4% ice-cold phosphate-buffered paraformaldehyde. Then the brain was quickly removed and fixed in 4% paraformaldehyde at 4° C. for about 4 hours before immersed sequentially in 20% and 30% PBS-buffered sucrose solution until sunk. Coronal sections (30 μm) were cut with a cryostat (Leica) and kept in the cryoprotectant buffer at −20° C. and subjected to immunostaining as described above.

Statistical analysis: Statistical analyses were performed in GraphPad. Significance was calculated with Student's t-test. The data are presented as the mean+/−SEM. *P<0.05; **P<0.01; ***P<0.001.

The invention has been described in connection with what are presently considered to be the most practical and preferred embodiments. However, this invention has been presented by way of illustration and is not intended to be limited to the disclosed embodiments. Accordingly, those skilled in the art will realize that the invention is intended to encompass all modifications and alternative arrangements within the spirit and scope of the invention as set forth in the appended claims.

We claim:

1. A method for expanding dopaminergic neuron progenitor cells comprising contacting the dopaminergic neuron progenitor cells with a culture medium comprising fibroblast growth factor 8b (FGF8b), an agonist of Hedgehog (Hh) signaling, a small-molecule agonist of canonical Wnt signaling, and WNT-C59, to generate an expanded dopaminergic neuron progenitor cell population.

2. The method of claim 1, wherein the agonist of Hh signaling is Smoothened agonist (SAG), SAG analog, SHH, SHH C25II, C24-SHH, purmorphamine, Hg—Ag, or derivatives thereof.

3. The method of claim 1, wherein the small-molecule agonist of canonical Wnt signaling is a glycogen synthase kinase 3 inhibitor.

4. The method of claim 3, wherein the glycogen synthase kinase 3 inhibitor is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, a lithium salt, or a combination thereof.

5. The method of claim 3, wherein the glycogen synthase kinase 3 inhibitor is CHIR99021 and is present in the culture medium at a concentration of about 0.01 micromolar (μM) to about 1 millimolar (mM).

6. The method of claim 5, wherein the CHIR99021 is present in the culture medium at a concentration of about 0.6 μM.

7. The method of claim 1, wherein WNT-C59 is present in the culture medium at a concentration of about 0.2 micromolar (μM) to about 2 μM.

8. The method of claim 7, wherein WNT-C59 is present in the culture medium at a concentration of about 0.5 μM.

9. The method of claim 1, wherein the dopaminergic neuron progenitor cells expand in vitro at least 300-fold.

10. The method of claim 1, wherein the culture medium further comprises neural supplement B27.

11. The method of claim 10, wherein the dopaminergic neuron progenitor cells expand in vitro at least 1000-fold.

12. The method of claim 1, wherein the culture medium comprises about 50 ng/ml FGF8b, about 25 ng/ml SHH, about 0.6 μM CHIR99021, about 0.5 μM WNT-C59.

13. The method of claim 1, wherein the dopaminergic neuron progenitor cells can be sub-cultured at least 6 times without loss of phenotype or genotype.

14. The method of claim 1, wherein the culture medium is chemically defined, serum-free, and xenogeneic material-free.

15. A substantially pure population of human dopaminergic neuron progenitor cells obtained according to the method of claim 1.

16. A composition comprising FGF8b, an agonist of Hh signaling, a small-molecule agonist of canonical Wnt signaling, and Wnt-C59.

17. The composition of claim 16, further comprising B27.

18. The composition of claim 16, wherein the small-molecule agonist of canonical Wnt signaling is a glycogen synthase kinase 3 inhibitor that is CHIR99021, 1-azakenpaullone, AR-A014418, indirubin-3′-monoxime, 5-Iodo-indirubin-3′-monoxime, kenpaullone, SB-415286, SB-216763, 2-anilino-5-phenyl-1,3,4-oxadiazole), (Z)-5-(2,3-Memylenedioxyphenyl)imidazolidine-2,4-dione, TWS119, CHIR98014, SB415286, Tideglusib, LY2090314, and a lithium salt, or a combination thereof.

19. The composition of claim 16, wherein the agonist of Hedgehog (Hh) signaling is Smoothened agonist (SAG), SAG analog, SHH, SHH C25II, C24-SHH, purmorphamine, Hg—Ag, or derivatives thereof.

20. The composition of claim 16, formulated as a cell culture medium.
